(19) United States
(12) Patent Application Publication
(10) Pub. No.: US 2023/0009472 A1
(43) Pub. Date: Jan. 12, 2023

(54) NEW COMPOSITION

(71) Applicant: Nanexa AB, Uppsala (SE)

(72) Inventors: Anders JOHANSSON, Uppsala (SE); Mårten ROOTH, Uppsala (SE); Joel HELLRUP, Uppsala (SE)

(21) Appl. No.: 17/782,564

(22) PCT Filed: Dec. 6, 2019
(86) PCT No.: PCT/GB2019/053453

§ 371 Date: Jun. 3, 2022

(51) Int. Cl. A61K 9/28; A61K 9/20; A61K 9/00; A61K 47/26; A61K 47/54; A61K 31/404

(52) U.S. Cl. CPC A61K 9/2813; A61K 9/0019; A61K 9/2086; A61K 31/404; A61K 47/26; A61K 47/549

(57) ABSTRACT

There is provided a composition comprising a plurality of particles of a weight-, number-, or volume-, based mean diameter that is between amount 10 nm and about 700 µm, which particles are made up of: (a) a solid core, which solid core preferably comprises a biologically active agent; (b) one or more discrete layers surrounding said core, said one or more layer s each comprising at least one separate coating material; and (c) a final overcoating layer of a coating material, which overcoating layer surrounds, encloses and/or encapsulates said core and said previously-applied layers of coating material, and which final layer is of a thickness that is less than said previously-applied layers. Said layers (b) and (c) are preferably applied by way of a gas phase coating technique, such as atomic layer deposition. When the cores comprise biologically active agent, the compositions may provide for the delayed or sustained release of said active agent without a burst effect.

### FIELD OF THE INVENTION

This invention relates to a new formulation for use in for example the field of drug delivery.

### PRIOR ART AND BACKGROUND

The listing or discussion of an apparently prior-published document in this specification should not necessarily be taken as an acknowledgement that the document is part of the state of the art or common general knowledge.

In the field of drug delivery, the ability to control the profile of drug release is of critical importance. It is desirable to ensure that active ingredients are released at a desired and predictable rate in vivo following administration, in order to ensure the optimal pharmacokinetic profile.

In the case of sustained release compositions, it is also of critical importance that a drug delivery composition provides a release profile that minimizes any initial rapid release of active ingredient, that is a large concentration of drug in plasma shortly after administration. Such a burst release may be hazardous in the case of drugs that have a narrow therapeutic window.

In the specific case of injectable suspensions, it is also important to ensure that the size of the suspended particles is controlled so that they can be injected through a needle. If large, aggregated particles are present, they will not only block the needle through which the suspension is to be injected, but also will not form a stable suspension within (i.e. they will instead tend to sink to the bottom of) the injection liquid.

There is thus a general need in the art for effective and/or improved drug transport and delivery systems.

Atomic later deposition (ALD) is a technique that is employed to deposit thin films comprising a variety of materials, including organic, biological, polymeric and, especially, inorganic materials, such as metal oxides, on solid substrates.

The technique is usually performed at low pressures and elevated temperatures. Film coatings are produced by alternating exposure of solid substrates within an ALD reactor chamber to vaporized reactants in the gas phase. Substrates can be silicon wafers, granular materials or small particles (e.g. microparticles or nanoparticles).

The coated substrate is protected from chemical reactions (decomposition) and physical changes by the solid coating. ALD can also potentially be used to control the rate of release of the substrate material within a solvent, which makes it of potential use in the formulation of active pharmaceutical ingredients.

In ALD, a first precursor, which can be metal-containing, is fed into an ALD reactor chamber (in a so called ‘precursor pulse’), and forms an adsorbed atomic or molecular monolayer at the surface of the substrate. Excess first precursor is then purged from the reactor, and then a second precursor, such as water, is pulsed into the reactor. This reacts with the first precursor, resulting in the formation of a monolayer of e.g. metal oxide on the substrate surface. A subsequent purging pulse is followed by a further pulse of the first precursor, and thus the start of a new cycle of the same events (a so called ‘ALD cycle’).

The thickness of the film coating is controlled by inter alia the number of ALD cycles that are conducted.

In a normal ALD process, because only atomic or molecular monolayers are produced during any one cycle, no discernible physical interface is formed between these monolayers, which essentially become a continuum at the surface of the substrate.

In international patent application WO 2014/187995, a process is described in which a number of ALD cycles are performed, which is followed by periodically removing the resultant coated substrates from the reactor and re-dispersion/agitation to present new surfaces available for precursor adsorption.

The agitation step is done primarily to solve a problem observed for nano- and microparticles, namely that, during the ALD coating process, aggregation of particles takes place, resulting in ‘pinholes’ being formed by contact points between such particles. The re-dispersion/agitation step was performed by placing the coated substrates in water and sonicating, which resulted in deagglomeration, and the breaking up of contact points between individual particles of coated active substance.

It has been found that the process of carrying out of ‘sets’ of ALD coating cycles followed by intermittent dispersion, as described in WO 2014/187995, results in clear, separate layers of coatings that are defined by clear, visible, physical interfaces between such coating layers. Such interfaces are clearly visible by a technique such as transmission electron microscopy (TEM) as regions of higher electron permeability. As explained below, similar interfaces are not visible when coatings are built up one atomic layer at a time from the surface of a substrate. This is the case even if different precursors are fed into the ALD reactor in consecutive ALD cycles.

We have now found that, after a final re-dispersion step, it is advantageous to provide a final, thinner ‘sealing’ shell of an inorganic coating material, as this enables the particles to be deagglomerated into primary particles without employing an aggressive deagglomeration technique such as sonication, so presenting them in a form that can be readily processed into a pharmaceutical formulation.

### DISCLOSURE OF THE INVENTION

According to a first aspect of the invention there is provided a composition in the form of a plurality of particles of a weight-, number-, and/or volume-based mean diameter that is between amount 10 nm and about 700 µm, which particles comprise (i.e. are made up of):
(a) solid cores, preferably comprising a biologically active agent;
(b) one or more discrete layer(s) surrounding (sequentially, in the case of more than one such layer) said cores, said one or more layer(s) each comprising at least one separate (i.e. separately applied) coating material; and
(c) an outer (i.e. final) overcoating layer of a coating material (a ‘sealing shell’), which overcoating layer surrounds, encloses and/or encapsulates said core and/or said previously-applied layer(s) of coating material, and which final layer is of a thickness that is less than said previously-applied layer(s),
which compositions are hereinafter referred to together as ‘the compositions of the invention’.

The term ‘solid’ will be well understood by those skilled in the art to include any form of matter that retains its shape and density when not confined, and/or in which molecules are generally compressed as tightly as the repulsive forces among them will allow. The solid cores have at least a solid exterior surface onto which a layer of coating material can be deposited. The interior of the solid cores may be also solid or may instead be hollow. For example, if the particles are spray dried before they are placed into the reactor vessel, they may be hollow due to the spray drying technique.

Compositions of the invention are preferably pharmaceutical compositions, in which case the composition may comprise a pharmacologically-effective amount of a biologically active agent. Furthermore, said solid cores preferably comprise said biologically active agent.

In this respect, the solid cores may consist essentially of, or comprise, biologically active agent (which agent may hereinafter be referred to interchangeably as a ‘drug’, and ‘active pharmaceutical ingredient (API)’ and/or an ‘active ingredient’). Biologically active agents also include biopharmaceuticals and/or biologics. Biologically active agents can also include a mixture of different APIs, as different API particles or particles comprising more than one API.

By ‘consists essentially’ of biologically-active agent, we include that the solid core is essentially comprised only of biologically active agent(s), i.e. it is free from non-biologically active substances, such as excipients, carriers and the like (vide infra). This means that the core may comprise less than about 5%, such as less than about 3%, including less than about 2%, e.g. less than about 1% of such other excipients.

In the alternative, cores comprising biologically active agent may include such an agent in admixture with one or more pharmaceutical ingredients, which may include pharmaceutically-acceptable excipients, such as adjuvants, diluents or carriers, and/or may include other biologically active ingredients.

Biologically active agents may be presented in a crystalline, a part-crystalline and/or an amorphous state. Biologically active agents may further comprise any substance that is in the solid state, orwhich may be converted into the solid state, at about room temperature (e.g. about 18° C.) and about atmospheric pressure, irrespective of the physical form. Such agents should also remain in the form of a solid whilst being coated in the reactor and also should not decompose physically or chemically to an appreciable agree (i.e. no more than about 10% w/w) whilst being coated, or after having been covered by at least one of the aforementioned layers of coating material. Biologically active agents may further be presented in combination (e.g. in admixture or as a complex) with another active substance.

As used herein, the term ‘biologically active agent’, or similar and/or related expressions, generally refer(s) to any agent, or drug, capable of producing some sort of physiological effect (whether in a therapeutic or prophylactic capacity against a particular disease state or condition) in a living subject, including, in particular, mammalian and especially human subjects (patients).

Biologically active agents may, for example, be selected from an analgesic, an anaesthetic, an anti-ADHD agent, an anorectics agent, an antiaddictives agent, an antibacterial agent, an antimicrobial agent, an antifungal agent, an antiviral agent, an antiparasitic agent, an antiprotozoal agent, an anthelminic, an ectoparasiticide, a vaccine, an anticancer agent, an antimetabolite, an alkylating agent, an antineoplastic agent, a topoisomerase, an immunomodulator, an immunostimulant, an immunosuppressant, an anabolic steroid, an anticoagulant agent, an antiplatelets agent, an anticonvulsant agent, an antidementia agent, an antidepressant agent, an antidote, an antihyperlipidemic agent, an antigout agent, an antimalarial, an antimigraine agent, an anti-inflammatory agent, an antiparkinson agent, an antipruritic agent, an antipsoriatic agent, an antiemetic, an anti-obesity agent, an anthelmintic, an anti-arrhythmic agent, an antiasthma agent, an antibiotic, an anticoagulant, an antidepressant, an antidiabetic agent, an antiepileptic, an antifibrinolytic agent, an antihemorrhagic agent, an antihistamine, an antitussive, an antihypertensive agent, an antimuscarinic agent, an antimycobacterial agent, an antioxidant agent, an antipsychotic agent, an antipyretic, an antirheumatic agent, an antiarrhythmic agent, an anxiolytic agent, an aphrodisiac, a cardiac glycoside, a cardiac stimulant, an entheogen, an entactogen, an euphoriant, an orexigenic, an antithyroid agent, an anxiolytic sedative, a hypnotic, a neuroleptic, an astringent, a bacteriostatic agent, a beta blocker, a calcium channel blocker, an ACE inhibitor, an angiotensin II receptor antagonist, a renin inhibitor, a beta-adrenoceptor blocking agent, a blood product, a blood substitute, a bronchodilator, a cardiac inotropic agent, a chemotherapeutic, a coagulant, a corticosteroid, a cough suppressant, a diuretic, a deliriant, an expectorant, a fertility agent, a sex hormone, a mood stabilizer, a mucolytic, a neuroprotective, a nootropic, a neurotoxin, a dopaminergic, an antiparkinsonian agent, a free radical scavenging agent, a growth factor, a fibrate, a bile acid sequestrants, a cicatrizant, a glucocorticoid, a mineralcorticoid, a haemostatic, a hallucinogen, a hypothalamic-pituitary hormone, an immunological agent, a laxative agent, a antidiarrhoeals agent, a lipid regulating agent, a muscle relaxant, a parasympathomimetic, a parathyroid calcitonin, a serenic, a statin, a stimulant, a wakefulness-promoting agent, a decongestant, a dietary mineral, a biphosphonate, a cough medicine, an ophthamological, an ontological, a H1 antagonist, a H2 antagonist, a proton pump inhibitor, a prostaglandin, a radiopharmaceutical, a hormone, a sedative, an anti-allergic agent, an appetite stimulant, an anoretic, a steroid, a sympathomimetic, a trombolytic, a thyroid agent, a vasodilator, a xanthine, an erectile dysfunction improvement agent, a gastrointestinal agent, a histamine receptor antagonist, a keratolytic, an antianginal agent, a non-steroidal antiinflammatory agent, a COX-2 inhibitor, a leukotriene inhibitor, a macrolide, a NSAID, a nutritional agent, an opioid analgesic, an opioid antagonist, a potassium channel activator, a protease inhibitor, an antiosteoporosis agent, a cognition enhancer, an antiurinary incontinence agent, a nutritional oil, an antibenign prostate hypertrophy agent, an essential fatty acid, a non-essential fatty acid, a radiopharmaceutical, a senotherapeutic, a vitamin, or a mixture of any of these.

The biologically-active agent may also be a cytokine, a peptidomimetic, a peptide, a protein, a toxoid, a serum, an antibody, a vaccine, a nucleoside, a nucleotide, a portion of genetic material, a nucleic acid, or a mixture thereof. Non-limiting examples of therapeutic peptides/proteins are as follows: lepirudin, cetuximab, dornase alfa, denileukin diftitox, etanercept, bivalirudin, leuprolide, alteplase, interferon alfa-n1, darbepoetin alfa, reteplase, epoetin alfa, salmon calcitonin, interferon alfa-n3, pegfilgrastim, sargramostim, secretin, peginterferon alfa-2b, asparaginase, thyrotropin alfa, antihemophilic factor, anakinra, gramicidin D, intravenous immunoglobulin, anistreplase, insulin (regular), tenecteplase, menotropins, interferon gamma-1b, interferon alfa-2a (recombinant), coagulation factor Vlla, oprelvekin, palifermin, glucagon (recombinant), aldesleukin, botulinum toxin Type B, omalizumab, lutropin alfa, insulin lispro, insulin glargine, collagenase, rasburicase, adalimumab, imiglucerase, abciximab, alpha-1-proteinase inhibitor, pegaspargase, interferon beta-1a, pegademase bovine, human serum albumin, eptifibatide, serum albumin iodinated, infliximab, follitropin beta, vasopressin, interferon beta-1b, hyaluronidase, rituximab, basiliximab, muromonab, digoxin immune Fab (ovine), ibritumomab, daptomycin, tositumomab, pegvisomant, botulinum toxin type A, pancrelipase, streptokinase, alemtuzumab, alglucerase, capromab, laronidase, urofollitropin, efalizumab, serum albumin, choriogonadotropin alfa, antithymocyte globulin, filgrastim, coagulation factor IX, becaplermin, agalsidase beta, interferon alfa-2b, oxytocin, enfuvirtide, palivizumab, daclizumab, bevacizumab, arcitumomab, eculizumab, panitumumab, ranibizumab, idursulfase, alglucosidase alfa, exenatide, mecasermin, pramlintide, galsulfase, abatacept, cosyntropin, corticotropin, insulin aspart, insulin detemir, insulin glulisine, pegaptanib, nesiritide, thymalfasin, defibrotide, natural alpha interferon/multiferon, glatiramer acetate, preotact, teicoplanin, canakinumab, ipilimumab, sulodexide, tocilizumab, teriparatide, pertuzumab, rilonacept, denosumab, liraglutide, golimumab, belatacept, buserelin, velaglucerase alfa, tesamorelin, brentuximab vedotin, taliglucerase alfa, belimumab, aflibercept, asparaginase erwinia chrysanthemi, ocriplasmin, glucarpidase, teduglutide, raxibacumab, certolizumab pegol, insulin isophane, epoetin zeta, obinutuzumab, fibrinolysin aka plasmin, follitropin alpha, romiplostim, lucinactant, natalizumab, aliskiren, ragweed pollen extract, secukinumab, somatotropin (recombinant), drotrecogin alfa, alefacept, OspA lipoprotein, urokinase, abarelix, sermorelin, aprotinin, gemtuzumab ozogamicin, satumomab pendetide, albiglutide, antithrombin alfa, antithrombin III (human), asfotase alfa, atezolizumab, autologous cultured chondrocytes, beractant, blinatumomab, C1 esterase inhibitor (human), coagulation factor XIII A-subunit (recombinant), conestat alfa, daratumumab, desirudin, dulaglutide, elosulfase alfa, evolocumab, fibrinogen concentrate (human), filgrastim-sndz, gastric intrinsic factor, hepatitis B immune globulin, human calcitonin, human clostridium tetani toxoid immune globulin, human rabies virus immune globulin, human Rho(D) immune globulin, human Rho(D) immune globulin, hyaluronidase (human, recombinant), idarucizumab, immune globulin (human), vedolizumab, ustekinumab, turoctocog alfa, tuberculin purified protein derivative, simoctocog alfa, siltuximab, sebelipase alfa, sacrosidase, ramucirumab, prothrombin complex concentrate, poractant alfa, pembrolizumab, peginterferon beta-1a, ofatumumab, obiltoxaximab, nivolumab, necitumumab, metreleptin, methoxy polyethylene glycol-epoetin beta, mepolizumab, ixekizumab, insulin degludec, insulin (porcine), insulin (bovine), thyroglobulin, anthrax immune globulin (human), anti-inhibitor coagulant complex, brodalumab, C1 esterase inhibitor (recombinant), chorionic gonadotropin (human), chorionic gonadotropin (recombinant), coagulation factor X (human), dinutuximab, efmoroctocog alfa, factor IX complex (human), hepatitis A vaccine, human varicella-zoster immune globulin, ibritumomab tiuxetan, lenograstim, pegloticase, protamine sulfate, protein S (human), sipuleucel-T, somatropin (recombinant), susoctocog alfa and thrombomodulin alfa.

Non-limiting examples of drugs which may be used according to the present invention are all-trans retinoic acid (tretinoin), alprazolam, allopurinol, amiodarone, amlodipine, asparaginase, astemizole, atenolol, azathioprine, azelatine, beclomethasone, bendamustine, bleomycin, budesonide, buprenorphine, butalbital, capecitabine, carbamazepine, carbidopa, carboplatin, cefotaxime, cephalexin, chlorambucil, cholestyramine, ciprofloxacin, cisapride, cisplatin, clarithromycin, clonazepam, clozapine, cyclophosphamide, cyclosporin, cytarabine, dacarbazine, dactinomycin, daunorubicin, diazepam, diclofenac sodium, digoxin, dipyridamole, divalproex, dobutamine, docetaxel, doxorubicin, doxazosin, enalapril, epirubicin, erlotinib, estradiol, etodolac, etoposide, everolimus, famotidine, felodipine, fentanyl citrate, fexofenadine, filgrastim, finasteride, fluconazole, flunisolide, fluorouracil, flurbiprofen, fluralaner, fluvoxamine, furosemide, gemcitabine, glipizide, gliburide, ibuprofen, ifosfamide, imatinib, indomethacin, irinotecan, isosorbide dinitrate, isotretinoin, isradipine, itraconazole, ketoconazole, ketoprofen, lamotrigine, lansoprazole, loperamide, loratadine, lorazepam, lovastatin, medroxyprogesterone, mefenamic acid, mercaptopurine, mesna, methotrexate, methylprednisolone, midazolam, mitomycin, mitoxantrone, moxidectine, mometasone, nabumetone, naproxen, nicergoline, nifedipine, norfloxacin, omeprazole, oxaliplatin, paclitaxel, phenyloin, piroxicam, procarbazine, quinapril, ramipril, risperidone, rituximab, sertraline, simvastatin, sulindac, sunitinib, temsirolimus, terbinafine, terfenadine, thioguanine, trastuzumab, triamcinolone, valproic acid, vinblastine, vincristine, vinorelbine, zolpidem, or pharmaceutically acceptable salts of any of these.

Compositions of the invention may comprise benzodiazipines, such as alprazomal, chlordiazepoxide, clobazam, clorazepate, diazepam, estazolam, flurazepam, lorazepam, oxazepam, quazepam, temazepam, triazolam and pharmaceutically acceptable salts of any of these.

Anaesthetics that may also be employed in the compositions of the invention may be local or general. Local anaesthetics that may be mentioned include amylocaine, ambucaine, articaine, benzocaine, benzonatate, bupivacaine, butacaine, butanilicaine, chloroprocaine, cinchocaine, cocaine, cyclomethycaine, dibucaine, diperodon, dimethocaine, eucaine, etidocaine, hexylcaine, fomocaine, fotocaine, hydroxyprocaine, isobucaine, levobupivacaine, lidocaine, mepivacaine, meprylcaine, metabutoxycaine, nitracaine, orthocaine, oxetacaine, oxybuprocaine, paraethoxycaine, phenacaine, piperocaine, piridocaine, pramocaine, prilocaine, primacaine, procaine, procainamide, proparacaine, propoxycaine, pyrrocaine, quinisocaine, ropivacaine, trimecaine, tolycaine, tropacocaine, or pharmaceutically acceptable salts of any of these.

Psychiatric drugs may also be employed in the compositions of the invention. Psychiatric drugs that may be mentioned include 5-HTP, acamprosate, agomelatine, alimemazine, amfetamine, dexamfetamine, amisulpride, amitriptyline, amobarbital, amobarbital/ secobarbital, amoxapine, amphetamine(s), aripiprazole, asenapine, atomoxetine, baclofen, benperidol, bromperidol, bupropion, buspirone, butobarbital, carbamazepine, chloral hydrate, chlorpromazine, chlorprothixene, citalopram, clomethiazole, clomipramine, clonidine, clozapine, cyclobarbital/diazepam, cyproheptadine, cytisine, desipramine, desvenlafaxine, dexamfetamine, dexmethylphenidate, diphenhydramine, disulfiram, divalproex sodium, doxepin, doxylamine, duloxetine, enanthate, escitalopram, eszopiclone, fluoxetine, flupenthixol, fluphenazine, fluspirilen, fluvoxamine, gabapentin, glutethimide, guanfacine, haloperidol, hydroxyzine, iloperidone, imipramine, lamotrigine, levetiracetam, levomepromazine, levomilnacipran, lisdexamfetamine, lithium salts, lurasidone, melatonin, melperone, meprobamate, metamfetamine, nethadone, methylphenidate, mianserin, mirtazapine, moclobemide, nalmefene, naltrexone, niaprazine, nortriptyline, olanzapine, ondansetron, oxcarbazepine, paliperidone, paroxetine, penfluridol, pentobarbital, perazine, pericyazine, perphenazine, phenelzine, phenobarbital, pimozide, pregabalin, promethazine, prothipendyl, protriptyline, quetiapine, ramelteon, reboxetine, reserpine, risperidone, rubidium chloride, secobarbital, selegiline, sertindole, sertraline, sodium oxybate, sodium valproate, sodium valproate, sulpiride, thioridazine, thiothixene, tianeptine, tizanidine, topiramate, tranylcypromine, trazodone, trifluoperazine, trimipramine, tryptophan, valerian, valproic acid in 2.3:1 ratio, varenicline, venlafaxine, vilazodone, vortioxetine, zaleplon, ziprasidone, zolpidem, zopiclone, zotepine, zuclopenthixol and pharmaceutically acceptable salts of any of these.

Opioid analgesics that may be employed in compositions of the invention include buprenorphine, butorphanol, codeine, fentanyl, hydrocodone, hydromorphone, meperidine, methadone, morphine, nomethadone, opium, oxycodone, oxymorphone, pentazocine, tapentadol, tramadol and pharmaceutically acceptable salts of any of these.

Opioid antagonists that may be employed in compositions of the invention include naloxone, nalorphine, niconalorphine, diprenorphine, levallorphan, samidorphan, nalodeine, alvimopan, methylnaltrexone, naloxegol, 6β-naltrexol, axelopran, bevenopran, methylsamidorphan, naldemedine, preferably nalmefeme and, especially, naltrexone, as well as pharmaceutically acceptable salts of any of these.

Anticancer agents that may be included in compositions of the invention include the following: actinomycin, afatinib, all-trans retinoic acid, amsakrin, anagrelid, arseniktrioxid, axitinib , azacitidine, azathioprine, bendamustine, bexaroten, bleomycin, bortezomib, bosutinib, busulfan, cabazitaxel, capecitabine, carboplatin, chlorambucil, cladribine, clofarabine, cytarabine, dabrafenib, dacarbazine, dactinomycin, dasatinib, daunorubicin, decitabine, docetaxel, doxifluridine, doxorubicin, epirubicin, epothilone, erlotinib, estramustin, etoposide, everolimus, fludarabine, fluorouracil, gefitinib, guadecitabine, gemcitabine, hydroxycarbamide, hydroxyurea, idarubicin, idelalisib, ifosfamide, imatinib, irinotecan, ixazomib, kabozantinib, karfilzomib, krizotinib, lapatinib, lomustin, mechlorethamine, melphalan, mercaptopurine, mesna, methotrexate, mitotan, mitoxantrone, nelarabin, nilotinib, niraparib, olaparib, oxaliplatin, paclitaxel, panobinostat, pazopanib, pemetrexed, pixantron, ponatinib, procarbazine, regorafenib, ruxolitinib, sonidegib, sorafenib, sunitinib, tegafur, temozolomid, teniposide, tioguanine, tiotepa, topotecan, trabektedin, valrubicin, vandetanib, vemurafenib, venetoklax, vinblastine, vincristine, vindesine, vinflunin, vinorelbine, vismodegib, as well as pharmaceutically acceptable salts of any of these. A preferred biologically active agent is azacitidine.

Such compounds may be used in any one of the following cancers: adenoid cystic carcinoma, adrenal gland cancer, amyloidosis, anal cancer, ataxia-telangiectasia, atypical mole syndrome, basal cell carcinoma, bile duct cancer, Birt-Hogg Dubé, tube syndrome, bladder cancer, bone cancer, brain tumor, breast cancer (including breast cancer in men), carcinoid tumor, cervical cancer, colorectal cancer, ductal carcinoma, endometrial cancer, esophageal cancer, gastric cancer, gastrointestinal stromal tumor, HER2-positive, breast cancer, islet cell tumor, juvenile polyposis syndrome, kidney cancer, laryngeal cancer, acute lymphoblastic leukemia, all types of acute lymphocytic leukemia, acute myeloid leukemia, adult leukemia, childhood leukemia, chronic lymphocytic leukemia, chronic myeloid leukemia, liver cancer, lobular carcinoma, lung cancer, small cell lung cancer, Hodgkin’s lymphoma, non-Hodgkin’s lymphoma, malignant glioma, melanoma, meningioma, multiple myeloma, myelodysplastic syndrome, nasopharyngeal cancer, neuroendocrine tumor, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, polycythemia vera, prostate cancer, renal cell carcinoma, retinoblastoma, salivary gland cancer, sarcoma, Kaposi sarcoma, skin cancer, small intestine cancer, stomach cancer, testicular cancer, thymoma, thyroid cancer, uterine (endometrial) cancer, vaginal cancer, Wilms’ tumor.

Other drugs that may be mentioned for use in compositions of the invention include immunomodulatory imide drugs, such as thalidomide and analogues thereof, such as pomalidomide, lenalidomide and apremilast, and pharmaceutically acceptable salts of any of these. Other drugs that many be mentioned include angiotensin II receptor type 2 agonists, such as Compound 21 (C21; 3-[4-(1H-imidazol-1-ylmethyl)phenyl]-5-(2-methylpropyl)thiophene-2-[(N-butyloxylcarbamate)-sulphonamide] and pharmaceutically acceptable (e.g. sodium) salts thereof.

Compositions of the invention may comprise a pharmacologically-effective amount of biologically-active agent. The term ‘pharmacologically-effective amount’ refers to an amount of such active ingredient, which is capable of conferring a desired physiological change (such as a therapeutic effect) on a treated patient, whether administered alone or in combination with another active ingredient. Such a biological or medicinal response, or such an effect, in a patient may be objective (i.e. measurable by some test or marker) or subjective (i.e. the subject gives an indication of, or feels, an effect), and includes at least partial alleviation of the symptoms of the disease or disorder being treated, or curing or preventing said disease or disorder.

Doses of active ingredients that may be administered to a patient should thus be sufficient to effect a therapeutic response over a reasonable and/or relevant timeframe. One skilled in the art will recognize that the selection of the exact dose and composition and the most appropriate delivery regimen will also be influenced by not only the nature of the active ingredient, but also inter alia the pharmacological properties of the formulation, the route of administration, the nature and severity of the condition being treated, and the physical condition and mental acuity of the recipient, as well as the age, condition, body weight, sex and response of the patient to be treated, and the stage/severity of the disease, as well as genetic differences between patients.

Administration of compositions of the invention may be continuous or intermittent (e.g. by bolus injection). Dosages of active ingredients may also be determined by the timing and frequency of administration.

In any event, the medical practitioner, or other skilled person, will be able to determine routinely the actual dosage of any particular active ingredient, which will be most suitable for an individual patient.

Alternatively, compositions as described herein may also comprise, instead of (or in addition to) biologically-active agents, diagnostic agents (i.e. agents with no direct therapeutic activity per se, but which may be used in the diagnosis of a condition, such as a contrast agents or contrast media for bioimaging).

Non-biologically active adjuvants, diluents and carriers that may be employed in cores to be coated in accordance with the invention may include pharmaceutically-acceptable substances that are soluble in water, such as carbohydrates, e.g. sugars, such as lactose and/or trehalose, and sugar alcohols, such as mannitol, sorbitol and xylitol; or pharmaceutically-acceptable inorganic salts, such as sodium chloride. Preferred carrier/excipient materials include sugars and sugar alcohols. Such carrier/excipient materials are particularly useful when the biologically active agent is a complex macromolecule, such as a peptide, a protein or portions of genetic material or the like, for example as described generally and/or the specific peptides/proteins described hereinbefore. Embedding complex macromolecules in excipients in this way will often result in larger cores for coating, and therefore larger coated particles, making it more beneficial to apply a sealing shell, which may comprise e.g. aluminium oxide.

It is not a requirement that the cores of the compositions of the invention comprise a biologically active agent. Whether the cores do or do not comprise a biologically active agent, the cores may comprise and/or consist essentially of a non-biologically active adjuvants, diluents and carriers, including emollients, and/or other excipients with a functional property, such as a buffering agent and/or a pH modifying agent (e.g. citric acid).

The cores are provided in the form of nanoparticles or, more preferably, microparticles. Preferred weight-, number-, or volume-, based mean diameters are between about 50 nm (e.g. about 100 nm, such as about 250 nm) and about 30 µm, for example between about 500 nm and about 100 µm, more particularly between about 1 µm and about 50 µm, such as about 25 µm, e.g. about 20 µm.

As used herein, the term ‘weight based mean diameter’ will be understood by the skilled person to include that the average particle size is characterised and defined from a particle size distribution by weight, i.e. a distribution where the existing fraction (relative amount) in each size class is defined as the weight fraction, as obtained by e.g. sieving (e.g. wet sieving). As used herein, the term ‘number based mean diameter’ will be understood by the skilled person to include that the average particle size is characterised and defined from a particle size distribution by number, i.e. a distribution where the existing fraction (relative amount) in each size class is defined as the number fraction, as measured by e.g. microscopy. As used herein, the term ‘volume based mean diameter’ will be understood by the skilled person to include that the average particle size is characterised and defined from a particle size distribution by volume, i.e. a distribution where the existing fraction (relative amount) in each size class is defined as the volume fraction, as measured by e.g. laser diffraction. Other instruments that are well known in the field may be employed to measure particle size, such as equipment sold by e.g. Malvern Instruments, Ltd (Worcestershire, UK) and Shimadzu (Kyoto, Japan).

Particles may be spherical, that is they possess an aspect ratio smaller than about 20, more preferably less than about 10, such as less than about 4, and especially less than about 2, and/or may possess a variation in radii (measured from the centre of gravity to the particle surface) in at least about 90% of the particles that is no more than about 50% of the average value, such as no more than about 30% of that value, for example no more than about 20% of that value.

Nevertheless, the coating of particles on any shape is also possible in accordance with the invention. For example, irregular shaped (e.g. ‘raisin’-shaped), needle-shaped, or cuboid-shaped particles may be coated. For a non-spherical particle, the size may be indicated as the size of a corresponding spherical particle of e.g. the same weight, volume or surface area. Hollow particles, as well as particles having pores, crevices etc., such as fibrous or ‘tangled’ particles may also be coated in accordance with the invention.

Particles may be obtained in a form in which they are suitable to be coated or be obtained in that form, for example by particle size reduction processes (e.g. crushing, cutting, milling or grinding to a specified weight based mean diameter (as hereinbefore defined), for example by wet grinding, dry grinding, air jet milling (including cryogenic micronization), ball milling, such as planetary ball milling, as well as making use of end-runner mills, roller mills, vibration mills, hammer mills, roller mill, fluid energy mills, pin mills, etc. Alternatively, particles may be prepared directly to a suitable size and shape, for example by spray-drying, precipitation, including the use of supercritical fluids or other top-down methods (i.e. reducing the size of large particles, by e.g. grinding, etc.), or bottom-up methods (i.e. increasing the size of small particles, by e.g. sol-gel techniques, etc.). Nanoparticles may alternatively be made by well-known techniques, such as gas condensation, attrition, chemical precipitation, ion implantation, pyrolysis, hydrothermal synthesis, etc.

It may be necessary (depending upon how the particles that comprise the cores are initially provided) to wash and/or clean them to remove impurities that may derive from their production, and then dry them. Drying may be carried out by way of numerous techniques known to those skilled in the art, including evaporation, spray-drying, vacuum drying, freeze drying, fluidized bed drying, microwave drying, IR radiation, drum drying, etc. If dried, cores may then be deagglomerated by grinding, screening, milling and/or dry sonication. Alternatively, cores may be treated to remove any volatile materials that may be absorbed onto its surface, e.g. by exposing the particle to vacuum and/or elevated temperature.

Surfaces of cores may be chemically activated prior to applying the first layer of coating material, e.g. by treatment with hydrogen peroxide, ozone, free radical-containing reactants or by applying a plasma treatment, in order to create free oxygen radicals at the surface of the core. This in turn may produce favourable adsorption/nucleation sites on the cores for the ALD precursors.

Preferably more than one layer of coating material is applied to the core sequentially. Preferred methods of applying the coating(s) to the cores comprising biologically active agents include gas phase techniques, such as ALD or related technologies, such as atomic layer epitaxy (ALE), molecular layer deposition (MLD; a similar technique to ALD with the difference that molecules (commonly organic molecules) are deposited in each pulse instead of atoms), molecular layer epitaxy (MLE), chemical vapor deposition (CVD), atomic layer CVD, molecular layer CVD, physical vapor deposition (PVD), sputtering PVD, reactive sputtering PVD, evaporation PVD and binary reaction sequence chemistry. ALD is the preferred method of coating according to the invention.

Preferably, more than one separate layers, coatings or shells (which terms are used herein interchangeably) are applied (that is ‘separately applied’) to the solid cores comprising biologically active agent. Such ‘separate application’ of ‘separate layers, coatings or subshells’ means that the solid cores are coated with a first layer of coating material, and then that resultant coated core is subjected to some form of deagglomeration process. In this respect, the number of discrete layers of coating material(s) as defined herein (also referred to as ‘subshells’) corresponds to the number of these intermittent deagglomeration steps, with a final deagglomeration step being conducted prior to the application of the outer overcoating later of coating material.

Coated cores may be subjected to the aforementioned deagglomeration process without being removed from said apparatus by way of a continuous process. Such a process will involve forcing solid product mass formed by coating said cores through a sieve that is located within the reactor, and is configured to deagglomerate any particle aggregates upon forcing of the coated cores by means of a forcing means applied within said reactor, prior to being subjected to a second and/or a further coating. This process is continued for as many times as is required and/or appropriate prior to the application of the final overcoating as described herein.

Having the sieve located within the reactor vessel means that the coating can be applied by way of a continuous process which does not require the particles to be removed from the reactor. Thus, no manual handling of the particles is required, and no external machinery is required to deagglomerate the aggregate particles. This not only considerably reduces the time of the coating process being carried out, but is also more convenient and reduces the risk of harmful (e.g. poisonous) materials being handled by personnel. It also enhances the reproducibility of the process by limiting the manual labour and reduces the risk of contamination.

Alternatively, coated cores may be removed from the coating apparatus, such as the ALD reactor, and thereafter subjected to an external deagglomeration step, for example as described in international patent application WO 2014/187995. Such an external deagglomeration step may comprise agitation, such as sonication in the wet or dry state, or preferably may comprise subjecting the resultant solid product mass that has been discharged from the reactor to sieving, e.g. by forcing it through a sieve or mesh in order to deagglomerate the particles, for example as described hereinafter, prior to placing the particles back into the coating apparatus for the next coating step. Again, this process may be continued for as many times as is required and/or appropriate prior to the application of the final overcoating as described herein.

In an external deagglomeration process, deagglomeration may alternatively be effected (additionally and/or instead of the abovementioned processes) by way of subjecting the coated particles in the wet or dry state to one or more of nozzle aerosol generation, milling, grinding, stirring, high sheer mixing and/or homogenization. If the step(s) of deagglomeration are carried out on particles in the wet state, the deagglomerated particles should be dried (as hereinbefore described in relation to cores) prior to the next coating step.

However, we prefer that, in such an external process, the deagglomeration step(s) comprise one or more sieving step(s), which may comprise jet sieving, manual sieving, vibratory sieve shaking, horizontal sieve shaking, tap sieving, or (preferably) sonic sifting as described hereinafter, or a like process, including any combination of these sieving steps.

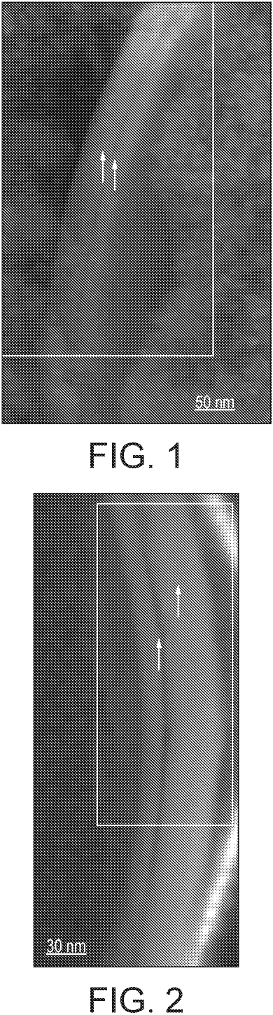

We have found that applying separate layers of coating materials following external deagglomeration gives rise to visible and discernible interfaces that may be observed by analysing coated particles according to the invention, and are observed by e.g. TEM as regions of higher electron permeability, for example, as can be seen in FIGS. 1 and 2.

This is to be contrasted to continuous ALD processes in which coated particles are not removed from the reactor prior to re-coating. Because, in an ALD coating process, coating takes place at the atomic level, even if different coating materials are sequentially employed (e.g. switching from one metal oxide precursor to another between ALD cycles), clear, physical interfaces, such as those shown in FIGS. 1 and 2 are not observed. Thus, the thickness of the layers between the interfaces that can be seen in FIGS. 1 and 2 correspond directly to the number of cycles in each series that are carried out within the ALD reactor, and between individual external agitation steps.

Without being limited by theory, it is believed that removing coated particles from the vacuum conditions of the ALD reactor and exposing a newly-coated surface to the atmosphere results in structural rearrangements due to relaxation and reconstruction of the outermost atomic layers. Such a process is believed to involve rearrangement of surface (and near surface) atoms, driven by a thermodynamic tendency to reduce surface free energy.

Furthermore, surface adsorption of species, e.g. hydrocarbons that are always present in the air, may contribute to this phenomenon, as can surface modifications, due to reaction of coatings formed with hydrocarbons, as well as atmospheric oxygen and the like. Accordingly, if such interfaces are analysed chemically, they may contain traces of contaminants that do not originate from the coating process, such as ALD.

Whether carried out inside or outside of the reactor, particle aggregates are preferably broken up by a forcing means that forces them through a sieve, thus separating the aggregates into individual particles or aggregates of a desired and predetermined size (and thereby achieving deagglomeration). In the latter regard, in some cases the individual primary particle size is so small (i.e. <1 µm) that achieving ‘full’ deagglomeration (i.e. where aggregates are broken down into individual particles) is not possible. Instead, deagglomeration is achieved by breaking down larger aggregates into smaller aggregates of secondary particles of a desired size, as dictated by the size of the sieve mesh. The smaller aggregates are then coated by the gas phase technique to form fully coated ‘particles’ in the form of small aggregate particles. In this way, the term ‘particles’, when referring the particles that have been deagglomerated and coated in the context of the invention, refers to both individual (primary) particles and aggregate (secondary) particles of a desired size.

In any event, the desired particle size (whether that be of individual particles or aggregates of a desired size) is maintained and, moreover, continued application of the gas phase coating mechanism to the particles after such deagglomeration via the sieve means that a complete coating is formed on the particle, thus forming fully coated particles (individual or aggregates of a desired size).

Whether carried out inside or outside of the reactor, the above-described repeated coating and deagglomeration process may be carried out at least 1, preferably 2, more preferably 3, such as 4, including 5, more particularly 6, e.g. 7 times, and no more than about 100 times, for example no more than about 50 times, such as no more than about 40 times, including no more than about 30 times, such as between 2 and 20 times, e.g. between 3 and 15 times, such as 10 times, e.g. 9 or 8 times, more preferably 6 or 7 times, and particularly 4 or 5 times.

The total thickness of the coating (meaning all the separate layers/coatings/shells) will on average be in the region of between about 0.5 nm and about 2 µm.

The minimum thickness of each individual subshell will on average be in the region of about 0.5 nm (for example about 0.75 nm, such as about 1 nm).

The maximum thickness of each individual subshell will depend on the size of the core (to begin with), and thereafter the size of the core with the coatings that have previously been applied, and may be on average about 1 hundredth of the mean diameter (i.e. the weight-, number-, or volume-, based mean diameter) of that core, or core with previously-applied coatings.

Preferably, for particles with a mean diameter that is between about 100 nm and about 1 µm, the subshell should be on average between about 1 nm and about 5 nm; for particles with a mean diameter that is between about 1 µm and about 20 µm, the coating thickness should be on average between about 1 nm and about 10 nm; for particles with a mean diameter that is between about 20 µm and about 700 µm, the coating thickness should be on average between about 1 nm and about 100 nm.

The thickness of the final, outer overcoating layer/coating, or sealing shell (which terms are used herein interchangeably), must be thinner than the subshells. The thickness may therefore be on average no more than a factor of about 0.7 (e.g. about 0.6) of the thickness of the widest previously-applied subshell. Alternatively, the thickness may be on average no more than a factor of about 0.7 (e.g. about 0.6) of the thickness of the last subshell that is applied, and/or may be on average no more than a factor of about 0.7 (e.g. about 0.6) of the average thickness of all of the previously-applied subshells. The thickness may be on average in the region of about 0.3 nm to about 10 nm, for particles up to about 20 µm. For larger particles, the thickness may be on average no more than about ⅟1000 of the coated particles’ weight-, number-, or volume-, based mean diameter.

We have found that applying subshells followed by conducting one or more deagglomeration step such as sonication gives rise to abrasions, pinholes, breaks, gaps, cracks and/or voids (hereinafter ‘cracks’) in the sub-shell coatings, due to coated particles essentially being more tightly ‘bonded’ or ‘glued’ together directly after the application of a thicker coating. This may expose a core comprising biologically-active ingredient to the elements once deagglomeration takes place.

The role of sealing shell is to provide a ‘sealing’ overcoating layer on the particles, covering over those cracks, so giving rise to particles that are not only completely covered by that sealing shell, but also covered in a manner that enables the particles to be deagglomerated readily (e.g. using a non-aggressive technique, such as vortexing) in a manner that does not destroy the subshells that have been formed underneath, prior to, and/or during, pharmaceutical formulation.

For example, if it is intended to provide a sample in suspension prior to administration to a patient, it is necessary to provide deagglomerated primary particles without pinholes or cracks in the coatings.

We have found that if a final, thinner sealing shell is not applied, it is often not possible to obtain an acceptable suspension for administration with adequately deagglomerated particles, unless an aggressive technique like sonication is applied. Such methods introduce the aforementioned cracks in the coatings, and/or samples where some shells are broken altogether. This will result in an undesirable initial peak (burst) in plasma concentration of active ingredient directly after administration.

Conversely, we have found that applying a thinner outer coating (sealing shell) allows the particles to be re-suspended in a solvent without such an aggressive deagglomeration step having been previously applied.

We have found instead that it is sufficient simply to subject the suspension to a less violent process, such as vortexing, stirring or mild sonication, and that this results in the deagglomerated coated particles with the essential absence of said cracks through which active ingredient can be released in an uncontrolled way. By ‘essentially free of said cracks’ in the coating(s), we mean that less than about 1% of the surfaces of the coated particles comprise abrasions, pinholes, breaks, gaps, cracks and/or voids through which active ingredient is potentially exposed (to, for example, the elements).

The subshells and thinner outer shell may, taken together, be of an essentially uniform thickness over the surface area of the particles. By ‘essentially uniform’ thickness, we mean that the degree of variation in the thickness of the inorganic coating of at least about 10%, such as about 25%, e.g. about 50%, of the coated particles that are present in a composition of the invention, as measured by TEM, is no more than about ±20%.

Coating materials that may be applied to cores may be pharmaceutically-acceptable, in that they should be essentially non-toxic.

Coating materials may comprise organic or polymeric materials, such as a polyamide, a polyimide, a polyurea, a polyurethane, a polythiourea, a polyesters or a polyimine. Coating materials may also comprise hybrid materials (as between organic and inorganic materials), including materials that are a combination between a metal, or another element, and an alcohol, a carboxylic acid, an amine or a nitrile. However, we prefer that coating materials comprise inorganic materials.

Inorganic coating materials may comprise one or more metals or metalloids, or may comprise one or more metal-containing, or metalloid-containing, compounds, such as metal, or metalloid, oxides, nitrides, sulphides, selenides, carbonates, and/or other ternary compounds, etc. Metal, and metalloid, hydroxides and, especially, oxides are preferred, especially metal oxides.

Metals that may be mentioned include alkali metals, alkaline earth metals, noble metals, transition metals, post-transition metals. Metal and metalloids that may be mentioned include aluminium, titanium, magnesium, iron, gallium, zinc, zirconium, niobium, hafnium, tantalum, lanthanum, and/or silicon; more preferably aluminium, titanium, magnesium, iron, gallium, zinc, zirconium, and/or silicon; especially aluminium, titanium and/or zinc.

As mentioned above, as the compositions of the invention comprises one or more discrete layers of inorganic coating materials, the nature and chemical composition(s) of those layers may differ from layer to layer.

Individual layers may also comprise a mixture of two or more inorganic materials, such as metal oxides or metalloid oxides, and/or may comprise multiple layers or composites of different inorganic or organic materials, to modify the properties of the layer.

Coating materials that may be mentioned include those comprising aluminium oxide (AI₂O₃), titanium dioxide (TiO₂), iron oxides (FeₓO_{y}, e.g. FeO and/or Fe₂O₃ and/or Fe₃O₄), gallium oxide (Ga₂O₃), magnesium oxide (MgO), zinc oxide (ZnO), niobium oxide (Nb₂O₅), hafnium oxide (HfO₂), tantalum oxide (Ta₂O₅), lanthanum oxide (La₂O₃), zirconium dioxide (ZrO₂) and/or silicon dioxide (SiO₂). Preferred coating materials include aluminium oxide, titanium dioxide, iron oxides, gallium oxide, magnesium oxide, zinc oxide, zirconium dioxide and silicon dioxide. More preferred coating materials include iron oxide, as well as titanium dioxide, zinc sulphide, zinc oxide and aluminium oxide.

Layers of coating materials (on an individual or a collective basis) in compositions of the invention may consist essentially (e.g. is greater than about 80%, such as greater than about, 90%, e.g. about 95%, such as about 98%) of iron oxides, aluminium oxide, zinc oxide or titanium dioxide. Coatings of zinc oxide (and iron oxides) may be thicker than corresponding coatings of aluminium oxide or titanium because they are more soluble. Accordingly, when the coating material(s) that is/are employed comprise e.g. zinc oxide, thicker coatings of materials may be employed, resulting in larger coated particles, making it more beneficial to apply a sealing shell, which may comprise the same material or a different material (e.g. aluminium oxide).

In ALD, layers of coating materials may be applied at process temperatures from about 20° C. to about 800° C., or from about 40° C. to about 200° C., e.g. from about 40° C. to about 150°, such as from about 50° C. to about 100° C. The optimal process temperature depends on the reactivity of the precursors and/or the substances (including biologically-active agents) that are employed in the core and/or melting point of the core substance(s).

In most instances, the first of the consecutive reactions will involve some functional group or free electron pairs or radicals at the surface to be coated, such as a hydroxy group (-OH) or a primary or secondary amino group (—NH₂ or —NHR where R e.g. is an aliphatic group, such as an alkyl group). The individual reactions are advantageously carried out separately and under conditions such that all excess reagents and reaction products are essentially removed before conducting the subsequent reaction.

Although the plurality of coated particles according to the invention are essentially free of the aforementioned cracks in the applied coatings, through which active ingredient is potentially exposed (to, for example, the elements), a further, optional step may be applied to the plurality of coated particles prior to subjecting it to further pharmaceutical formulation processing. This optional step may comprise ensuring that the few remaining particles with broken and/or cracked shells/coatings are subjected to a treatment in which all particles are suspended in a solvent in which the active ingredient is soluble (e.g. with a solubility of at least about 1 mg/mL), but the least soluble material in the coating is insoluble (e.g. with a solubility of no more than about 0.1 µg/mL), followed by separating solid matter particles from solvent by, for example, centrifugation, sedimentation, flocculation and/or filtration, resulting in mainly intact particles being left.

The above-mentioned optional step provides a means of potentially reducing further the likelihood of a (possibly) undesirable initial peak (burst) in plasma concentration of active ingredient, as discussed hereinbefore.

At the end of the process, coated particles may be dried using one or more of the techniques that are described hereinbefore for drying cores. Drying may take place in the absence, or in the presence, of one or more pharmaceutically acceptable excipients (e.g. a sugar or a sugar alcohol).

Alternatively, at the end of the process, separated particles may be resuspended in a solvent (e.g. water, with or without the presence of one or more pharmaceutically acceptable excipients as defined herein), for subsequent storage and/or administration to patients.

Prior to applying the first layer of coating material or between successive coatings, cores and/or partially coated particles may be subjected to one or more alternative and/or preparatory surface treatments. In this respect, one or more intermediary layers comprising different materials (i.e. other than the inorganic material(s)) may be applied to the relevant surface, e.g. to protect the cores or partially-coated particles from unwanted reactions with precursors during the coating step(s)/deposition treatment, to enhance coating efficiency, or to reduce agglomeration.

An intermediary layer may, for example, comprise one or more surfactants, with a view to reducing agglomeration of particles to be coated and to provide a hydrophilic surface suitable for subsequent coatings. Suitable surfactants in this regard include well known non-ionic, anionic, cationic or zwitterionic surfactants, such as the Tween series, e.g. Tween 80. Alternatively, cores may be subjected to a preparatory surface treatment if the active ingredient that is employed as part of (or as) that core is susceptible to reaction with one or more precursor compounds that may be present in the gas phase during the coating (e.g. the ALD) process.

Application of ‘intermediary’ layers/surface treatments of this nature may alternatively be achieved by way of a liquid phase non-coating technique, followed by a lyophilisation, spray drying or other drying method, to provide particles with surface layers to which coating materials may be subsequently applied.

Outer surfaces of particles of compositions of the invention may also be derivatized or functionalized, e.g. by attachment of one or more chemical compounds or moieties to the outer surfaces of the final layer of coating material, e.g. with a compound or moiety that enhances the targeted delivery of the particles within a patient to whom the nanoparticles are administered. Such a compound may be an organic molecule (such as PEG) polymer, an antibody or antibody fragment, or a receptor-binding protein or peptide, etc.

Alternatively, the moiety may be an anchoring group such as a moiety comprising a silane function (see, for example, Herrera et al, J. Mater. Chem., 18, 3650 (2008) and US 8,097,742). Another compound, e.g. a desired targeting compound may be attached to such an anchoring group by way of covalent bonding, or non-covalent bonding, including bonding, hydrogen bonding, or van der Waals bonding, or a combination thereof.

The presence of such anchoring groups may provide a versatile tool for targeted delivery to specific sites in the body. Alternatively, the use of compound such as PEG may cause particles to circulate for a longer duration in the blood stream, ensuring that they do not become accumulated in the liver or the spleen (the natural mechanism by which the body eliminates particles, which may prevent delivery to diseased tissue).

Compositions of the invention are either suitable for administration to patients as they are prepared (i.e. as a plurality of particles) or are preferably formulated together with one or more pharmaceutically-acceptable excipients, including adjuvants, diluents or carriers for use in the medicinal or veterinary fields (including in therapy and/or, if the core comprises a diagnostic material, in diagnostics).

There is further provided compositions of the invention for use in medicine, diagnostics, and/or in veterinary practice and a pharmaceutical (or veterinary) formulation comprising a composition of the invention and a pharmaceutically- (or veterinarily-) acceptable adjuvant, diluent or carrier.

Compositions of the invention may be administered locally, topically or systemically, for example orally (enterally), by injection or infusion, intravenously or intraarterially (including by intravascular or other perivascular devices/dosage forms (e.g. stents)), intramuscularly, intraosseously, intracerebrally, intracerebroventricularly, intrasynovially, intrasternally, intrathecally, intralesionally, intracranially, intratumorally, cutaneously, intracutaneous, subcutaneously, transmucosally (e.g. sublingually or buccally), rectally, transdermally, nasally, pulmonarily (e.g. by inhalation, tracheally or bronchially), topically, or by any other parenteral route, such as subcutaneously or intramuscularly, optionally in the form of a pharmaceutical (or veterinary) preparation comprising the compound in a pharmaceutically (or veterinarily) acceptable dosage form.

The incorporation of compositions of the invention into pharmaceutical formulations may be achieved with due regard to the intended route of administration and standard pharmaceutical practice. Pharmaceutically acceptable excipients, such as carriers may be chemically inert to the biologically-active agent and may have no detrimental side effects or toxicity under the conditions of use. Such pharmaceutically acceptable carriers may also impart an immediate, or a modified, release of compositions of the invention.

Pharmaceutical (or veterinary) formulations comprising compositions of the invention may include particles of different types, for example particles comprising different active ingredients, comprising different functionalization (as described hereinbefore), particles of different sizes, and/or different thicknesses of the layers of coating materials, or a combination thereof. By combining, in a single pharmaceutical formulation, particles with different coating thicknesses and/or different core sizes, the drug release following administration to patient may be controlled (e.g. varied or extended) over a specific time period.

For peroral administration (i.e. administration to the gastrointestinal tract by mouth with swallowing), compositions of the invention may be formulated in a variety of dosage forms. Pharmaceutically acceptable carriers or diluents may be solid or liquid. Solid preparations include granules (in which granules may comprise some or all of the plurality of particles of a composition of the invention in the presence of e.g. a carrier and other excipients, such as a binder or pH adjusting agents), compressed tablets, pills, lozenges, capsules, cachets, etc. Carriers include materials that are well known to those skilled in the art, including those disclosed hereinbefore in relation to the formulation of biologically active agents within cores, as well as magnesium carbonate, pectin, dextrin, starch, gelatin, tragacanth, methylcellulose, sodium carboxymethylcellulose, a low melting wax, cocoa butter, lactose, microcrystalline cellulose, low-crystalline cellulose, and the like.

Solid dosage forms may comprise further excipients, such as flavouring agents, lubricants, binders, preservatives, disintegrants, and/or encapsulating materials. For example, compositions of the invention may be encapsulated e.g. in a soft or hard shell capsule, e.g. a gelatin capsule.

Compositions of the invention formulated for rectal administration, which may include suppositories that may contain, for example, a suitable non-irritating excipient, such as cocoa butter, synthetic glyceride esters or polyethylene glycols, which are solid at ordinary temperatures, but which liquefy and/or dissolve in the rectal cavity to release the particles of the compositions of the invention.

For parenteral administration, such as subcutaneous and/or intramuscular injections, the compositions of the invention may be in the form of sterile injectable and/or infusible dosage forms, for example, sterile aqueous or oleaginous suspensions of compositions of the invention.

Such suspensions may be formulated in accordance with techniques that are well known to those skilled in the art, by employing suitable dispersing or wetting agents (e.g. Tweens, such as Tween 80), and suspending agents.

Non-toxic parenterally-acceptable diluents also include solutions of 1,3-butanediol, mannitol, Ringer’s solution, isotonic sodium chloride solution, sterile, fixed oils (including any bland fixed oil, such as synthetic mono- or diglycerides). Fatty acids, such as oleic acid and its glyceride derivatives may be used in the preparation of injectable formulations, as well as natural pharmaceutically-acceptable oils, such as olive oil or castor oil, and their polyoxyethylated versions, and pH adjusting agents. These oil suspensions may also contain a long-chain alcohol diluent or dispersant.

Compositions of the invention suitable for injection may also comprise compositions in the form of a liquid, a sol or a gel (e.g. comprising hyaluronic acid), which is administrable via a surgical administration apparatus, e.g. a needle, a catheter or the like, to form a depot formulation. The use of compositions of the invention may control the dissolution rate and the pharmacokinetic profile by reducing any burst effect as hereinbefore defined and/or by increasing the length of release of biologically active ingredient from that formulation.

Compositions of the invention may also be formulated for inhalation, e.g. as an inhalation powder for use with a dry powder inhaler (see, for example, those described by Kumaresan et al, Pharma Times, 44, 14 (2012) and Mack et al., Inhalation, 6, 16 (2012)), the relevant disclosures thereof are hereby incorporated by reference. Suitable particle sizes for the plurality of particles in a composition of the invention for use in inhalation to the lung are in the range of about 2 to about 10 µm.

Compositions of the invention may also be formulated for administration topically to the skin, or to a mucous membrane. For topical application, the pharmaceutical formulations may be provided in the form of e.g. a lotion, a gel, a paste, a tincture, a transdermal patch, a gel for transmucosal delivery, all of which may comprise a composition of the invention. The composition may also be formulated with a suitable ointment containing a composition of the invention suspended in a carrier, such as a mineral oil, liquid petroleum, white petroleum, propylene glycol, polyoxyethylene polyoxypropylene compound, emulsifying wax or water. Suitable carrier for lotions or creams include mineral oils, sorbitan monostearate, polysorbate 60, cetyl esters wax, cetaryl alcohol, 2-octyldodecanol, benzyl alcohol and water.

Pharmaceutical formulations may comprise between about 1% to about 99%, such as between about 10% (such as about 20%, e.g. about 50%) to about 90% by weight of the composition of the invention, with the remainder made up by pharmaceutically acceptable excipients.

In any event, compositions of the invention, may be formulated with conventional pharmaceutical additives and/or excipients used in the art for the preparation of pharmaceutical formulations, and thereafter incorporated into various kinds of pharmaceutical preparations and/or dosage forms using standard techniques (see, for example, Lachman et al, ‘The Theory and Practice of Industrial Pharmacy’, Lea & Febiger, 3^{rd} edition (1986); ‘Remington: The Science and Practice of Pharmacy’, Troy (ed.), University of the Sciences in Philadelphia, 21^{st} edition (2006); and/or ‘Aulton’s Pharmaceutics: The Design and Manufacture of Medicines’, Aulton and Taylor (eds.), Elsevier, 4^{th} edition, 2013), and the documents referred to therein, the relevant disclosures in all of which documents are hereby incorporated by reference. Otherwise, the preparation of suitable formulations may be achieved non-inventively by the skilled person using routine techniques.

Wherever the word ‘about’ is employed herein, for example in the context of amounts (e.g. concentrations, dimensions (sizes and/or weights), size ratios, aspect ratios, proportions or fractions), temperatures or pressures, it will be appreciated that such variables are approximate and as such may vary by ±15%, such as ±10%, for example ±5% and preferably ±2% (e.g. ±1%) from the numbers specified herein. This is the case even if such numbers are presented as percentages in the first place (for example ‘about 15%’ may mean ±15% about the number 10, which is anything between 8.5% and 11.5%).

Compositions of the invention allow for the formulation of a large diversity of pharmaceutically active compounds. Compositions of the invention may be used to treat effectively a wide variety of disorders depending on the biologically active agent that is included.

Compositions of the invention may further be formulated in the form of injectable suspension of coated particles with a size distribution that is both even and capable of forming a stable suspension within the injection liquid (i.e. without settling) and may be injected through a needle.

Furthermore, compositions of the invention may provide a release and/or pharmacokinetic profile that minimizes any burst effect, which is characterised by a concentration maximum shortly after administration.

The compositions and processes described herein may have the advantage that, in the treatment of a relevant condition with a particular biologically active agent, they may be more convenient for the physician and/or patient than, be more efficacious than, be less toxic than, have a broader range of activity than, be more potent than, produce fewer side effects than, or that it may have other useful pharmacological properties over, any similar treatments that may be described in the prior art for the same active ingredient.

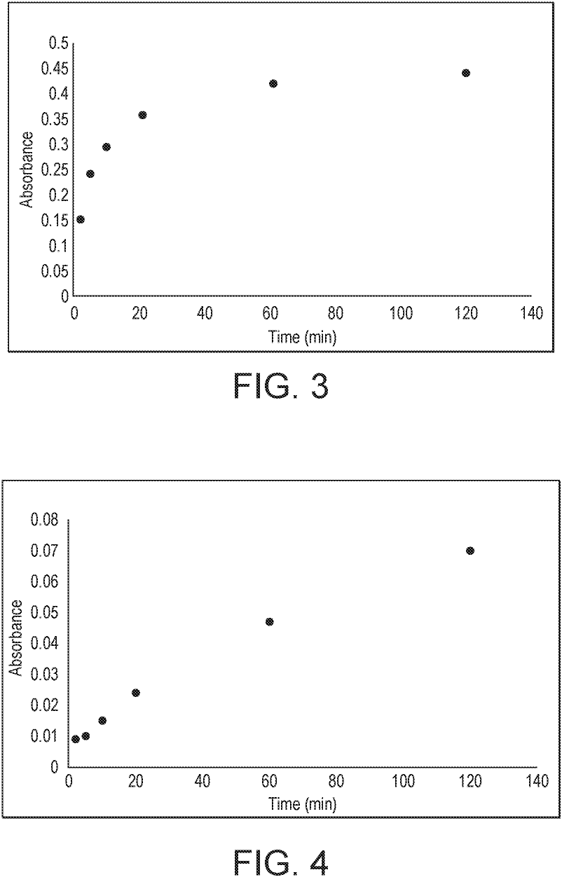

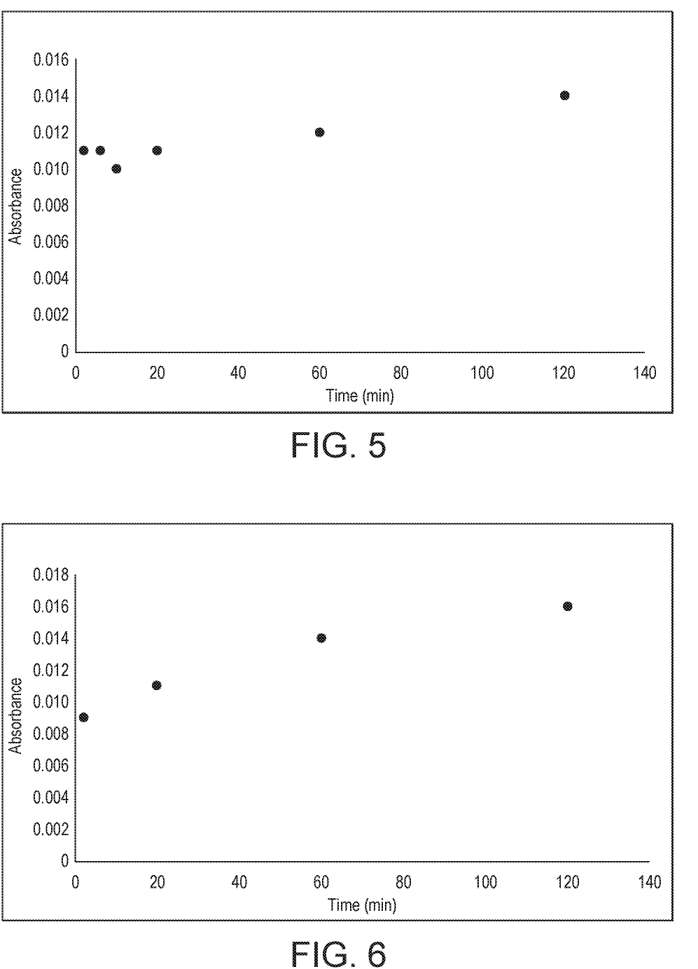

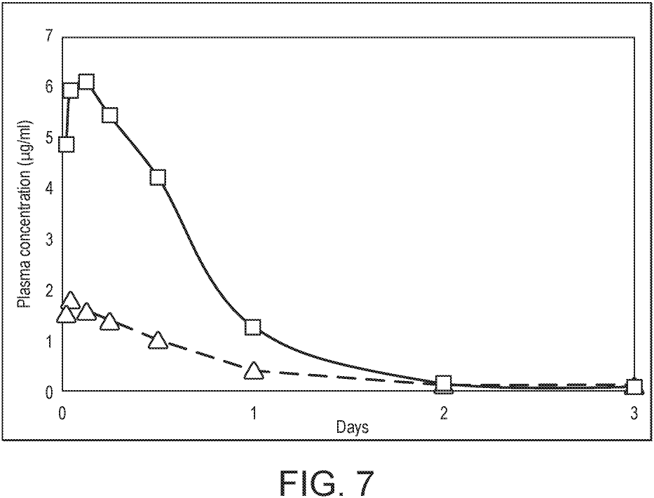

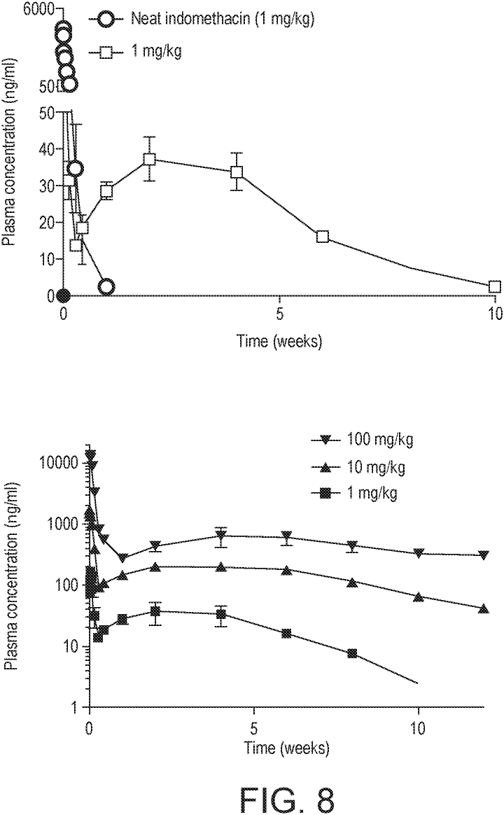

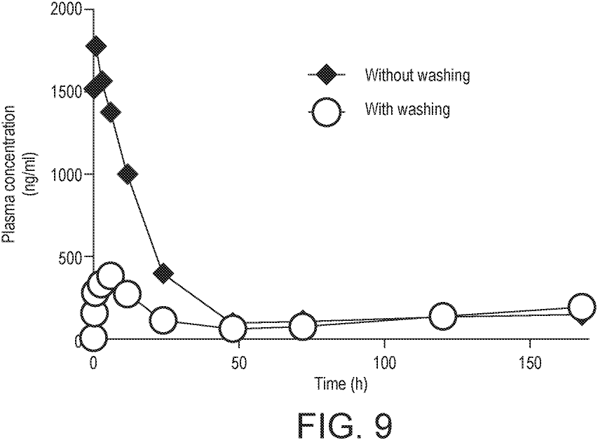

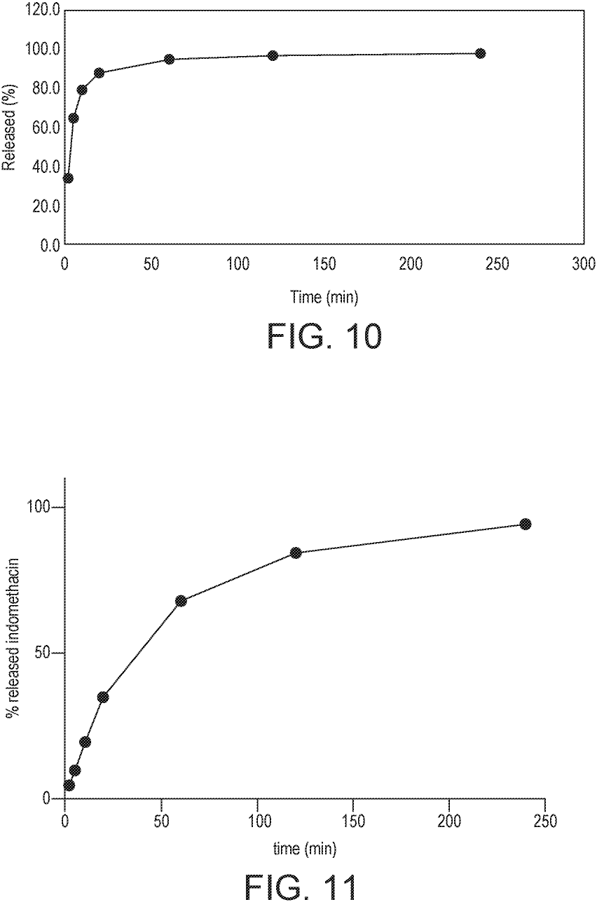

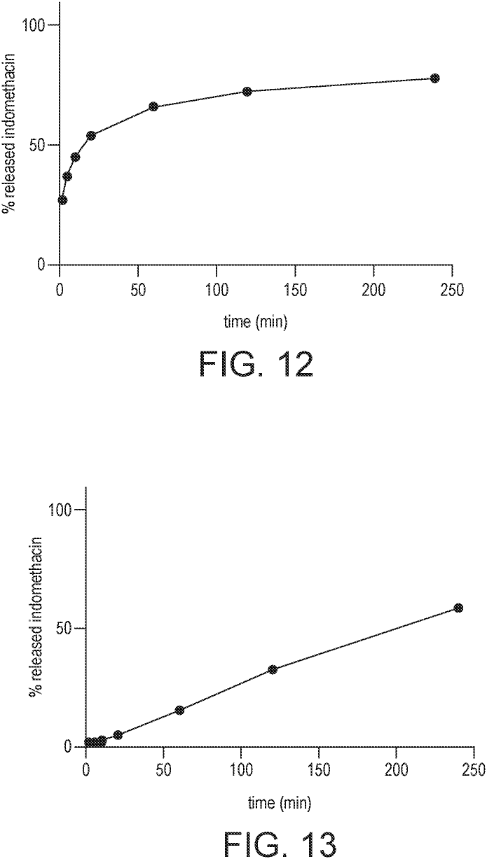

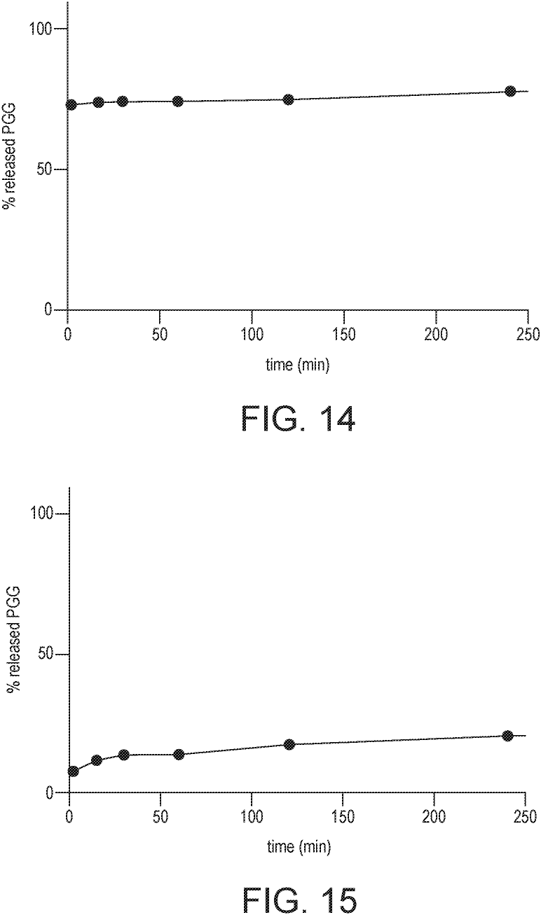

The invention is illustrated, but in no way limited, by the following examples with reference to the attached figure in which: FIGS. 1 and 2 are TEM images that show clearly visible physical interfaces (regions of higher electron permeability) that are formed by employing the process that is described herein; FIGS. 3 and 4 show in vitro indomethacin release from aluminium oxide coated particles prior to (FIG. 3) and after (FIG. 4) application of a sealing shell; FIG. 5 shows indomethacin release from particles with a sealing shell also subjected to a final washing step; FIG. 6 shows indomethacin release from particles subjected to a final washing step but without a sealing shell; FIG. 7 shows a comparison between in vivo plasma concentration-time profiles in rats injected with coated indomethacin particles with (white squares) and without (black triangles) the presence of a sealing shell; FIG. 8 shows a comparison between in vivo plasma concentration-time profiles in rats injected with coated indomethacin particles at different doses; FIG. 9 shows a comparison between in vivo plasma concentration-time profiles in rats injected with the same dose of coated indomethacin particles made by the same process, with and without being subjected to a final washing step; FIGS. 10 and 11 show in vitro indomethacin release from coated particles with zinc oxide subshells prior to (FIG. 10) and after (FIG. 11) application of an aluminium oxide sealing shell; FIGS. 12 and 13 show in vitro indomethacin release from coated particles with titanium oxide subshells prior to (FIG. 12) and after (FIG. 13) application of an aluminium oxide sealing shell; and FIGS. 14 and 15 show in vitro phenylalanine-glycine-glycine tripeptide release from coated particles prior to (FIG. 14) and after (FIG. 15) application of a sealing shell.

### EXAMPLES

### Example 1

### Coated Indomethacin Microparticles I

Microparticles of indomethacin (Hangzhou APIChem Technology Co. Ltd., China) were prepared by wet ball milling (Fritsch, Premium line, Pulverisette 7, IDar-Oberstein, Germany). The mean diameter of the ball milled indomethacin particles was 5.9 µm as determined by laser diffraction (Shimadzu, SALD-7500nano, Kyoto, Japan).

After milling, the suspension was washed and dried to form a powder consisting of the indomethacin microparticles. The dried microparticles were dispersed using dry sieving (100 µm mesh).

The powder was loaded to an ALD reactor (Picosun, SUNALE™ R-series, Espoo, Finland). 15 ALD cycles were performed at a reactor temperature of 50° C. Trimethyl aluminium and water were used as precursors, forming a first subshell of aluminium oxide. The first subshell was about 4 to 5 nm in thickness (as estimated from the number of ALD cycles).

The powder was extracted from the reactor and deagglomerated by means of sieving 100 µm mesh sieve followed by sieving 20 µm mesh.

The powder was loaded to the ALD reactor. 15 ALD cycles were performed at a reactor temperature of 50° C. Trimethyl aluminium and water were used as precursors, forming a second subshell of aluminium oxide. The second subshell was about 4 to 5 nm in thickness (estimated from the number of ALD cycles).

The powder was extracted from the reactor and deagglomerated by means of sieving, first with a 100 µm mesh sieve, and then with a 20 µm mesh sieve. The degree of deagglomeration was measured by means of laser diffraction and the average particle diameter was measured as 6 µm.

The coating-deagglomeration steps were repeated twice further to form third and fourth subshells with the same thicknesses on the particles.

40 mg of powder was placed in a test tube and 3 mL of a dispersing solution comprising water with 0.5% of Tween-80 (Merck, Kenilworth, NJ, USA) was added. The suspension was gently vortexed (Vortex-Genie 2 (Scientific Industries Ltd., New York, USA)) for 1 minute and the particle size distribution was measured by means of laser diffraction. The average particle diameter was measured as 6 µm.

The suspension was added to a dissolution bath with 1 L of phosphate buffer solution (pH 7.2, 25 mM, 37° C.). Samples were withdrawn from the bath at 2, 5, 10, 20, 60 and 120 minutes and filtered through a 0.2 µm filter. The filtered samples were analyzed for indomethacin content using an UV/vis spectrometer (Ultrospec 2100 pro (Amersham Biosciences, Little Chalfont, UK)) operating at a wavelength of 320 nm. The release of indomethacin, as determined by absorbance against time, is plotted in FIG. 3.

After drying, the powder loaded to the ALD reactor. 10 further ALD cycles were performed at a reactor temperature of 50° C., using trimethyl aluminium and water as precursors, forming a ‘sealing’ shell of aluminium oxide. The resultant sealing shell was about 3 nm in thickness (as estimated from the number of ALD cycles).

40 mg of the resultant powder was placed in a test tube and 3 mL of the same dispersion solution was added. The suspension was vortexed for 1 minute and the degree of deagglomeration measured by means of laser diffraction. The average particle diameter was measured as 6 µm.

The suspension was added to a dissolution bath with 1 L of phosphate buffer solution (pH 7.2, 25 mM, 37° C.). Samples were withdrawn from the bath at 2, 5, 10, 20, 60 and 120 minutes, filtered through a 0.2 µm filter. The filtered sample was analyzed for indomethacin content using the UV/vis spectrometer operating at 320 nm. Again, release of indomethacin over time is plotted in FIG. 4.

A clear difference between the release profiles as between FIGS. 3 and 4 can be seen, with that shown in FIG. 4 being much slower. This suggests that, after applying a sealing shell, the subshells are intact to a higher degree, with less indomethacin being exposed to the dissolution solution.

### Example 2

### Coated Indomethacin Microparticles II

The sample from Example 1 was washed placing 40 mg of the sample into a test tube and adding 10 mL of dimethyl sulfoxide. The suspension so formed was vortexed for 1 minute and then centrifuged at 7000 x g (Biofuge primo R (Heraeus, Hanau, Germany)) for 5 minutes. The solvent was decanted and the wet powder was kept in the test tube.
10 mL of 99.7% ethanol was added and the vortexing, centrifuging and decanting step repeated.
3 mL of the same dispersing solution identified in Example 1 was added. The suspension was gently vortexed for 1 minute and the degree of deagglomeration was measured by means of laser diffraction. The average particle diameter was measured as 6 µm.

The suspension was added to a dissolution bath with 1 L of phosphate buffer solution (pH 7.2, 25 mM, 37° C.). Samples were withdrawn as in Example 1. The release profile is shown in FIG. 5.

By comparing FIG. 4 with FIG. 5, it can be seen that the release of indomethacin is even slower when the sample has been subjected to the above-mentioned washing and suggests that indomethacin from the particles with cracks has been removed during the washing and left only particles with intact and dense shells in the sample.

### Example 3

### Comparison Between Particle With and Without Sealing Shells

A sample with sealing shell was prepared as described in Example 1 but without the final steps applying the sealing shell. Washing, dissolution and analysis of that sample was performed as described in Example 2 and the release profile is presented in FIG. 6.

Comparing the release profiles between FIGS. 5 and 6 shows that the indomethacin content in the washed samples is higher in the sample with a sealing shell. The amount of indomethacin that is ‘wasted’ is about 4 times higher for the sample without the sealing shell (15.5% versus 4%).

### Example 4

### In Vivo Rat Model I

Samples similar to those described in Example 1 (four subshells without sealing shell) were suspended in a 0.5% Tween-80 solution. A second suspension was prepared with a sample with four subshells with a sealing shell as described earlier.

Both suspensions were injected subcutaneously in the dorsal region of male Sprague-Dawley rats and plasma samples were extracted. Indomethacin content was measured by means of HPLC-MS/MS (Xeco TDS-micro (Waters, Milford, MA, USA)), using a diode array detector set at 254 nm (Shimadzu). Separation was accomplished with a phenylhexyl column, 4.6*150 mm, 2.6 µm particle size in a column oven set at 40° C. Mobile phase A was 10 g/L acetic acid in water and mobile phase B was acetonitrile. A gradient elution program was applied, with mobile phase A decreasing from 70 to 30%. Quantification was carried out with a straight-line equation from a six-point calibration curve, ranging from 0.5-100 µg/mL. Run time for the analysis was 31 minutes.

The solid line and square points in FIG. 7 show the plasma concentration profile from this first test group and the dotted line and triangular points show the plasma concentration profile from the second test group.

The peaks in the diagram represents the initial burst release of indomethacin the first hours after administration of the suspensions. It can be seen that the sealing shell on the particles of the first suspension reduce the burst release by approximately a factor of 4.

### Example 5

### In Vivo Rat Model II

Samples were prepared according to the method described in Example 1 (four subshells and a sealing shell).

Suspensions were injected subcutaneously (in doses of 1, 10 and 100 mg/kg body weight (BW), with 6 rats in each group) in the dorsal region of male Sprague-Dawley rats, and compared with an injection of neat indomethacin (1 mg/kg BW). Plasma samples were extracted at different times. Indomethacin content was analysed by means of HPLC-MS/MS (Xeco TDS-micro (Waters, Milford, MA, USA)).

The results from the plasma sample analysis can be seen in FIG. 8. The left hand plasma concentration time profile shows the comparison between neat indomethacin and the 1 mg/kg coated sample. The right hand profile shows a comparison between the different coated samples. The plasma concentration-time profile of nanoshell coated indomethacin demonstrated sustained release over 12 weeks when administered subcutaneously at 10 or 100 mg/kg. Neat indomethacin was fully eliminated within one week.

### Example 6

### In Vivo Rat Model III

Samples were prepared according to the method described in Example 2 (four subshells and a sealing shell and thereafter washed).

Suspensions of the samples were used in the rat model described in Example 5 (10 mg/kg BW).

The results from the plasma sample analysis of this study can be seen in FIG. 9. For comparison FIG. 9 also includes the 10 mg/kg results from Example 5.

From the plasma concentration profile it can be seen that washing of the samples significantly reduce the initial burst of drug release.

### Example 7

### Coated Indomethacin Microparticles III

Microparticles of indomethacin were prepared, washed, dried and dispersed as described in Example 1 above.

The powder was loaded to the same ALD reactor as described in Example 1. 15 ALD cycles were performed at a reactor temperature of 50° C., using diethyl zinc (DEZ) and water as precursors, forming a first subshell of zinc oxide. The first subshell was about 4 to 5 nm in thickness (estimated from the number of ALD cycles).

The powder was extracted from the reactor and deagglomerated by means of sonic sifter (Tsutsui Scientific SW-20AT, China) using a nylon sieve with 20 µm mesh (Tsutsui Scientific, China).

The powder was once again loaded to the ALD reactor and 15 further ALD cycles performed forming a second subshell of zinc oxide. The second subshell was estimated as being about 4 to 5 nm in thickness.

The powder was extracted from the reactor and deagglomerated using the sonic sifter as described above.

The coating-deagglomeration steps were repeated twice further to form third and fourth subshells with the same thicknesses on the particles to form a sample without sealing shell.

The total indomethacin content in the produced sample without sealing shell was measured by HPLC as described in Example 4 above.

41.09 and 39.74 mg (duplicate testing) of the sample were placed into test tubes. 3 mL of the dispersing solution was added and the suspensions vortexed as described in Example 1.

The two suspensions were added to dissolution baths, and samples withdrawn and filtered through a 0.2 µm filter as described in Example 1. The filtered samples were analyzed for indomethacin content as described in Example 1. Mean values (with standard deviation; the error bars cannot be seen since the analytical data are almost identical) for release of indomethacin from the two samples are plotted in FIG. 10.

The sample was re-loaded back into the ALD reactor and a further 15 ALD cycles performed at a reactor temperature of 50° C., using trimethyl aluminium and water as precursors, forming a sealing shell of aluminium oxide. The resultant sealing shell was about 4 to 5 nm in thickness (as estimated from the number of ALD cycles).

The total indomethacin content in the produced sample with a sealing shell was measured by HPLC as described in Example 4 above.

40.41 and 40.18 mg (duplicate testing) of the sample with the sealing shell were placed in a test tubes, 3 mL of the dispersion solution added and the suspensions vortexed as described above.

The suspensions were then added to dissolution baths and the release of indomethacin analyzed as described above, as plotted in FIG. 11.

A clear difference between the release profiles as between FIGS. 10 and 11 can be seen, with the release shown in FIG. 11 being much slower. This suggests that, after applying a sealing shell of aluminium oxide onto four subshells of zinc oxide, the subshells are intact to a much higher degree, with less indomethacin being exposed to the dissolution solution.

### Example 8

### Coated Indomethacin Microparticles IV

The same procedure as that described in Example 7 above was repeated but, instead of DEZ, titanium tetrachloride was employed to provide four separate subshells of titanium dioxide.

Release profiles for indomethacin are show in FIG. 12 (without an aluminium oxide sealing shell) and FIG. 13 (with a sealing shell). A difference can be seen, with that shown in FIG. 13 being slower in the beginning, gives a much lower burst release in vitro. Again, this suggests that, after applying a sealing shell of aluminium oxide onto four subshells of titanium dioxide, the subshells are intact to a higher degree, with less indomethacin being exposed to the dissolution solution.

### Example 9

### Coated Peptide Microparticles

Microparticles comprising trehalose and phenylalanine-glycine-glycine (PGG) (both Sigma-Aldrich Co. St. Louis, USA) were prepared by spray-drying using a Mini Spray Dryer B-290 (Büchi, Switzerland).

130 mL of a solution of PGG (0.5%), trehalose (9.7%) and Tween 80® (0.2%) in water. This resulted in microparticles comprising 4.7% PGG. The spray-drying was performed with an inlet temperature of 125° C., a pump flow of 4.2 mL/min, and a resulting outlet temperature of 73° C.

The microparticle powder was loaded to an ALD reactor (Picosun, SUNALE™ R-series, Espoo, Finland). 25 ALD cycles were performed at a reactor temperature of 50° C. Trimethyl aluminium and water were used as precursors, forming a first subshell of aluminium oxide. The first subshell was about 7 to 8 nm in thickness (estimated from the number of ALD cycles).

The powder was extracted from the reactor and deagglomerated using a sonic sifter as described in Example 7 above.

Three further coating-deagglomeration steps were performed to provide four subshells in total with the same thicknesses.

The total PGG content in the sample so produced was measured by HPLC as described in Example 4 above.

199.22 mg of the sample was then placed in a test tube, 3 mL of the dispersing solution added, the suspension vortexed for 1 minute, added to a dissolution bath, and indomethacin release measured, as described in Example 1 above.

PGG released was analysed with HPLC and is plotted in FIG. 14.

The remaining sample was loaded back into the ALD reactor and a further 15 ALD cycles performed as described above forming a ‘sealing’ shell of aluminium oxide (about 4 to 5 nm in thickness, as estimated from the number of ALD cycles).

The total PGG content in the produced sample with sealing shell was measured by HPLC.

199.12 and 200.28 mg of powder (duplicate testing) were added to test tubes and 3 mL of the dispersing solution added, followed by gentle vortexing of the resultant suspension for 1 minute, addition of the suspension to a dissolution bath, and analysis of PGG content released conducted, as described above. Release of PGG against time is plotted (mean of two values) in FIG. 15.

A clear difference between the release profiles as between FIGS. 14 and 15 can be seen, with that shown in FIG. 15 being much slower, again suggesting that the sealing shell onto four subshells renders them intact to a higher degree, with less indomethacin being exposed to the dissolution solution.

1. A composition in the form of a plurality of particles of a weight-, number-, or volume-based mean diameter that is between amount 10 nm and about 700 µm, which particles comprise:
(a) solid cores;
(b) one or more discrete layers surrounding said cores, said one or more layers each comprising at least one separate coating material; and
(c) an outer overcoating layer of a coating material, which overcoating layer surrounds, encloses and/or encapsulates said core and/or said previously-applied layers of coating material, and which final layer is of a thickness that is less than said previously-applied layers.

2. A composition as claimed in claim 1, wherein the cores comprise a biologically active agent and/or a pharmaceutically-acceptable excipient.

3. A composition as claimed in claim 2, wherein the carrier/excipient material is a sugar or a sugar alcohol and/or is a pH modifying agent.

4. A composition as claimed in claim 1, wherein the cores consist essentially of biologically active agent.

5. A composition as claimed in claim 4, wherein the biologically active agent is selected from an analgesic, an anaesthetic, an anti-ADHD agent, an anorectics agent, an antiaddictives agent, an antibacterial agent, an antimicrobial agent, an antifungal agent, an antiviral agent, an antiparasitic agent, an antiprotozoal agent, an anthelminic, an ectoparasiticide, a vaccine, an anticancer agent, an antimetabolite, an alkylating agent, an antineoplastic agent, a topoisomerase, an immunomodulator, an immunostimulant, an immunosuppressant, an anabolic steroid, an anticoagulant agent, an antiplatelets agent, an anticonvulsant agent, an antidementia agent, an antidepressant agent, an antidote, an antihyperlipidemic agent, an antigout agent, an antimalarial, an antimigraine agent, an anti-inflammatory agent, an antiparkinson agent, an antipruritic agent, an antipsoriatic agent, an antiemetic, an anti-obesity agent, an anthelmintic, an anti-arrhythmic agent, an antiasthma agent, an antibiotic, an anticoagulant, an antidepressant, an antidiabetic agent, an antiepileptic, an antifibrinolytic agent, an antihemorrhagic agent, an antihistamine, an antitussive, an antihypertensive agent, an antimuscarinic agent, an antimycobacterial agent, an antioxidant agent, an antipsychotic agent, an antipyretic, an antirheumatic agent, an antiarrhythmic agent, an anxiolytic agent, an aphrodisiac, a cardiac glycoside, a cardiac stimulant, an entheogen, an entactogen, an euphoriant, an orexigenic, an antithyroid agent, an anxiolytic sedative, a hypnotic, a neuroleptic, an astringent, a bacteriostatic agent, a beta blocker, a calcium channel blocker, an ACE inhibitor, an angiotensin II receptor antagonist, a renin inhibitor, a beta-adrenoceptor blocking agent, a blood product, a blood substitute, a bronchodilator, a cardiac inotropic agent, a chemotherapeutic, a coagulant, a corticosteroid, a cough suppressant, a diuretic, a deliriant, an expectorant, a fertility agent, a sex hormone, a mood stabilizer, a mucolytic, a neuroprotective, a nootropic, a neurotoxin, a dopaminergic, an antiparkinsonian agent, a free radical scavenging agent, a growth factor, a fibrate, a bile acid sequestrants, a cicatrizant, a glucocorticoid, a mineralcorticoid, a haemostatic, a hallucinogen, a hypothalamic-pituitary hormone, an immunological agent, a laxative agent, a antidiarrhoeals agent, a lipid regulating agent, a muscle relaxant, a parasympathomimetic, a parathyroid calcitonin, a serenic, a statin, a stimulant, a wakefulness-promoting agent, a decongestant, a dietary mineral, a biphosphonate, a cough medicine, an ophthamological, an ontological, a H1 antagonist, a H2 antagonist, a proton pump inhibitor, a prostaglandin, a radio-pharmaceutical, a hormone, a sedative, an anti-allergic agent, an appetite stimulant, an anoretic, a steroid, a sympathomimetic, a trombolytic, a thyroid agent, a vasodilator, a xanthine, an erectile dysfunction improvement agent, a gastrointestinal agent, a histamine receptor antagonist, a keratolytic, an antianginal agent, a non-steroidal antiinflammatory agent, a COX-2 inhibitor, a leukotriene inhibitor, a macrolide, a NSAID, a nutritional agent, an opioid analgesic, an opioid antagonist, a potassium channel activator, a protease inhibitor, an anti osteoporosis agent, a cognition enhancer, an antiurinary incontinence agent, a nutritional oil, an antibenign prostate hypertrophy agent, an essential fatty acid, a non-essential fatty acid, a cytokine, a peptidomimetic, a peptide, a protein, a radiopharmaceutical, a senotherapeutic, a toxoid, a serum, an antibody, a nucleoside, a nucleotide, a vitamin, a portion of genetic material, a nucleic acid, or a mixture of any of these.

6. A composition as claimed in claim 1, wherein the weight-, number-, or volume-, based mean diameter of the cores is between amount 1 µm and about 50 µm.

7. A composition as claimed in claim 1, wherein, not including the final overcoating, more than one discrete layers of coating material is applied to the core sequentially.

8. A composition as claimed in claim 7, wherein between 3 and 10 discrete layers of coating material are applied.

9. A composition as claimed in claim 1, wherein, not including the overcoating layer, the total thickness of the discrete layers of coating material is between about 0.5 nm and about 2 µm.

10. A composition as claimed in claim 1, wherein the maximum thickness of an individual discrete layer of coating material is about 1 hundredth of the weight-, number-, or volume-based mean diameter of the core, including any other previously-applied discrete layers of coating material that are located between said individual discrete layer and the outer surface of the core.

11. A composition as claimed in claim 1, wherein the thickness of the overcoating layer is no more than a factor of about 0.7 of the thickness of the widest previously-applied discrete coating.

12. A composition as claimed in claim 11, wherein, for particles up to about 20 µm, the thickness of the overcoating layer is between about 0.3 nm to about 10 nm.

13. A composition as claimed in claim 1, wherein the thickness of the overcoating layer is no more than about ⅟1000 of the weight-, number-, or volume-based mean diameter of the core, including any discrete layers of coating material.

14. A composition as claimed in claim 1, wherein the overcoating layer gives rise to particles that are essentially free of abrasions, pinholes, breaks, gaps, cracks and/or voids through which active ingredient, if present, is potentially exposed.

15. A composition as claimed in claim 1, wherein the coating materials of the one or more discrete layers and/or overcoating layer comprise one or more inorganic materials.

16. A composition as claimed in claim 15, wherein the coating materials comprise one or more metal-containing, or metalloid-containing, compounds.

17. A composition as claimed in claim 16, wherein the compounds comprise a hydroxide and/or an oxide.

18. A composition as claimed in claim 16, wherein the compounds comprise aluminium oxide, titanium dioxide and/or zinc oxide.

19. A composition as claimed in claim 1, wherein the coating material of the overcoating layer comprises aluminium oxide.

20. A composition as defined in claim 1 for use in medicine or in veterinary practice.

21. A pharmaceutical or veterinary formulation comprising a composition as defined in claim 1 and a pharmaceutically-acceptable or a veterinarily-acceptable adjuvant, diluent or carrier.

22. A formulation as claimed claim 21 in the form of a sterile injectable and/or infusible dosage form.

23. A formulation as claimed claim 22 in the form of a liquid, a sol or a gel, administrable via a surgical administration apparatus that forms a depot formulation.

24. A process for the preparation of a composition as defined in claim 1, which comprises applying layers of coating materials to cores, and/or previously-coated cores, by atomic layer deposition.

25. A process as claimed in claim 24, wherein:
(i) solid cores are coated with a first discrete layer of coating material;
(ii) the coated cores from step (i) are then subjected to a deagglomeration process step;
(iii) the deagglomerated coated cores from step (ii) are then coated with a second discrete layer of coating material;
(iv) repeating steps (ii) and (iii) to obtain the required number of discrete layers;
(v) subjecting the coated particles from step (iv) to a final deagglomeration process step; and
(vi) application of the final overcoating layer of coating material to the deagglomerated coated particles from step (v).

26. A process as claimed in claim 25, wherein the deagglomeration step that takes place between applications of coatings comprises sieving.

27. A process as claimed in claim 26, wherein the sieving comprises sonic sifting.

28. A process as claimed in claim 24, wherein the particles are subjected to a final vortexing step after application of the overcoating layer.

29. A process as for the preparation of a formulation as defined in claim 21, which comprises admixing a composition as defined in claim 1 with the relevant pharmaceutically-acceptable or a veterinarily-acceptable adjuvant, diluent or carrier.

30. A process as claimed in claim 24, which process comprises an further step of resuspending separated particles in a solvent, with or without the presence of one or more pharmaceutically acceptable excipients.
